(19) United States
(12) Patent Application Publication
(10) Pub. No.: US 2018/0112277 A1
(43) Pub. Date: Apr. 26, 2018

(54) SIGNAL DIRECTED DISSECTION TO INFORM CANCER THERAPY STRATEGY

(71) Applicant: EXPRESSION PATHOLOGY, INC., Rockville, MD (US)

(72) Inventor: David KRIZMAN, Gaithersburg, MD (US)

(21) Appl. No.: 15/795,046

(22) Filed: Oct. 26, 2017

Related U.S. Application Data
(60) Provisional application No. 62/413,371, filed on Oct. 26, 2016.

(51) Int. Cl. C12Q 1/68; A61K 39/395; G01N 33/574

(52) U.S. Cl. CPC C12Q 1/6886; A61B 2017/00761; G01N 33/57492; A61K 39/39558

(57) ABSTRACT

A novel signal directed tissue microdissection method is provided that forms the foundation for an entire multistep panomic (proteomic/genomic) process to inform and ascertain an optimal individualized cancer treatment strategy. Patient tumor tissue is sectioned onto DIRECTOR slides and tumor cells are identified by an inclusion signal while unwanted cells such as normal stroma are identified by an exclusion signal, and whereby such signals are determined by a clinically-trained histologist/pathologist, the presence or absence of immunohistochemical staining, or a combination of both. A liquefied biochemical lysate is prepared from the tumor cells whereby genomics and proteomics assays are performed to inform optimal cancer treatment strategies for the patient that includes chemotherapy agents, targeted therapeutic agents, cancer vaccines, and immunomodulatory agents individually or in combination.

### CROSS-REFERENCE TO RELATED APPLICATIONS

This application claims priority to provisional application Serial No. 62/413,371, filed Oct. 26, 2016, the contents of which are hereby incorporated by reference in their entirety.

### FIELD OF INVENTION

The field of invention is tissue microdissection, which is the selective and precise collection of specified populations of cells directly from thin sections of pathologically processed solid tissue. The tissue is most notably surgically-removed diseased tissue from a patient suffering from a disease such as cancer. The purpose of collecting specified populations of cells from said tissue is to understand the molecular nature of the diseased cells thus informing the treatment strategy for the patient from which the tissue was removed and pathologically processed.

### BACKWARDS

The background description includes information that may be useful in understanding the present disclosed subject matter. It is not an admission that any of the information provided herein is prior art or relevant to the presently disclosed subject matter, or that any publication specifically or implicitly referenced is prior art.

A novel tissue microdissection method, termed Signal Directed Dissection, is described and is the central focal point of a multistep process for informing optimal cancer treatment strategy, or strategies, for an individual cancer patient. Signal Directed Dissection selectively and precisely collects specific cells and homogeneous populations of cells identified for molecular analysis directly from heterogeneous cancer patient tumor tissue. A tissue section from heterogeneous patient tumor tissue is sectioned onto a DIRECTOR slide, whereby specified and desired cells to be excised for molecular analysis, such as tumor cells, are identified by standard histological methods and digitally marked with a colorimetric signal termed an inclusion signal. The non-desirable cells that should not be excised from heterogeneous cancer patient tissue and that should not be analyzed are identified by standard histological methods and digitally marked using a uniquely different color termed an exclusion signal. The presence of both inclusion and exclusion signals in the same tissue section provides for greater precision by sharpening the boundaries between cells to be transferred out from the tissue and cells to remain within the tissue, ultimately leading to increased purity of collected cellular populations. Cells in patient tumor tissue that will most often be analyzed are malignant tumor cells and cells that will most often not be analyzed are benign cells such as stromal, normal epithelial, and lymphocytic cells.

Activation of a laser is induced by the presence of the inclusion signal while the laser is prevented from activating by presence of the exclusion signal. When an inclusion signal is encountered, a laser beam whose width is measured in approximately 1-15 μM in diameter impacts the DIRECTOR slide at each and every μM of area that is identified by the inclusion signal. When the laser strikes the DIRECTOR slide at precisely an inclusion signal an explosive event takes place by virtue of contact of the photon energy with the energy transfer coating resulting in laser-induced forward transfer of a cell, or cells, downward into a receiving vesicle. When the laser is prevented from striking the DIRECTOR slide at a point of an exclusion signal there is no downward transfer of the cells into a receiving vesicle in which case the cells remain in the tissue. In this way, there is precise and efficient collection in the receiving vesicle of only the desired cells, most often the tumor cells, identified by an inclusion signal. Optimal cancer treatment strategies seek out and eradicate only the tumor cells from the patient and thus basing tumor cell killing on the molecular makeup of the tumor cells is the most effective way in which to administer that strategy. The method describing the use of a DIRECTOR slide for laser induced forward transfer of tissue via utilization of an energy transfer interlayer coating is described in U.S. Pat. No. 7,381,440, the contents of which are hereby incorporated by reference in their entirety.

Inclusion and exclusion signals are determined via evaluation of stained histology by a clinically-trained histologist/pathologist, the presence or absence of colorimetric signal induced by a chemical reaction based on immunohistochemical and/or in situ hybridization methods, or various combinations. These approaches for determining signal are simply listed by way of example and do not describe all the various ways in which inclusion and exclusion signals can be generated. A digital image of the tissue containing inclusion signal, exclusion signal, or both is prepared in standard digital imaging format (e.g., PNG, PDF, TIFF, JPEG, GIFF, SVS, etc.) thus digitally imprinting the information about which cells within patient tumor tissue should or should not be collected. The digital image with digitally imprinted inclusion/exclusion signals is then utilized by special computer software to control laser activation whereby a laser is activated to impact the energy transfer coating of the DIRECTOR slide only at points of the digitally imprinted inclusion signal while the laser is prevented by the software from activating when the digitally imprinted exclusion signal is encountered. The process of laser induced forward transfer of cells from tissue using the presently described method comprises the following:

1) developing a digital image wherein specific cells/cell populations have been identified by inclusion/exclusion signals,

2) uploading the digital image into a computer,

3) placing the DIRECTOR slide containing the tissue from which the signal-containing digital image was developed into a slide holder that resides within a tissue microdissection instrument comprising, in order, a laser positioned above said DIRECTOR slide where the tissue is mounted on the opposite side of the energy transfer coating from the laser and a receiving vesicle directly below the tissue,

4) directing the software to activate the laser to strike the energy transfer coating of the DIRECTOR slide at precisely the points of only inclusion signal thus transferring the cells/cell populations identified by inclusion signal downward into a receiving vesicle, and

5) direct the software to prevent activation of the laser at points of exclusion signal so those cells/cell populations identified by exclusion signal remain on the slide within the tissue.

Molecular analysis of patient tissue samples, both normal and diseased, benefits greatly from the ability to procure pure homogenous populations of cells directly from pathologically defined tissue sections where tremendous amounts of cellular heterogeneity can exist. Tumor tissue comprises many types of cells including tumor epithelial cells, normal epithelial cells, fibroblastic connective cells, and immune cells. In order to understand the status of the tumor cells only, there needed to be a way in which just the tumor cells could be removed from the heterogeneous mixture of cells and studied. This is the reasoning behind development of the field of tissue microdissection as originally introduced and patented in 1995 by the Laser Capture Microdissection (LCM) technology (e.g., see U.S. Pat. No. 6,867,038). LCM as well as other tissue microdissection technologies have improved the analysis of tissue samples by providing a means through which molecular profiling of cells derived from tissue samples can be placed in a pathologically relevant context.

Other tissue microdissection technologies in addition to LCM have been developed and are commercially available. However, none of these technologies rely on laser-induced forward transfer of cells via digitally identified and imprinted inclusion and exclusion signals. These other technologies have led to the commercial availability of tissue microdissection instruments including the PixCell systems (see Arcturus), the PALM system (see PALM Microlaser Technologies), the uCuT (see Molecular Machines and Industries), the Leica AS LMD (see Leica Microsystems), the LaserScissors (see Cell Robotics), the MicroDissector (see Eppendorf), xMD (see xMDx), and the Clonis system (see Bio-Rad). These techniques generally use one of two methods. The first is a contact method whereby a thin film is placed on top of and in contact a section of the tumor tissue so that when a single laser event is used to illuminate through the film from the top, it activates the film to become adherent to the tissue. When the film is subsequently pulled off the tissue, the cells of interest are stuck to the underside of the film and the cells of interest are then removed from the film by biochemical procurement methods (see U.S. Patents 62/51,467, 62/51,516, 68/67,038). The second method utilizes a glass slide coated with a polyethylene, polyethylene-naphthalene, polyester, polyacrylate polymer (PET/PEN) that contains at least 5% by weight of an aromatic or part-aromatic polycondensate whereby the coating is situated in between the glass slide and the tissue. A primary laser event is used to cut the polymer coating around the cells of interest in order to isolate and separate the cells of interest from the surrounding cells. This is then followed by a subsequent second laser illumination event to catapult the separated cells along with the separate PET/PEN coating upward into a collection vessel. This method utilizes two separate laser illumination events (see U.S. Pat. No. 5,998,129). An additional method similarly utilizes the same polyethylene terephthalate (PET) or polyethylene naphthalate (PEN) coating between the tissue to be dissected and the glass slide; however, this approach relies on laser light to cut the tissue and the PET/PEN coating around the cells of interest after which the process of gravity causes the PET/PEN coating along with the region containing the cells of interest that has been isolated by laser etching to simply fall downwards into a collection vessel (not patented). Yet another approach that utilizes the PET/PEN coated glass slides is performed whereby a laser is used from below to cut a circle around the cells of interest thereby also cutting the PET coating to isolate the cells of interest from the surrounding tissue. An adhesive film is then lowered onto the tissue coming into contact with the regions that were cut. The cells of interest are then isolated away from section by pulling the cap upwards which contains the cells that were cut out from the surrounding tissue (see US Published Patent Application 2011/0181866).

Still another recent adhesive film-based method, termed Expression Microdissection (xMD), relies on staining a tissue section using IHC followed by layering a light activated film on top of and in contact with the tissue. When a white light is shined on the film there is an interaction between the chemical residue from the IHC reaction and the film. Based on this interaction the cells of interest that are IHC positive are stuck to the underside of the film and the cells of interest are then removed from the film by biochemical procurement methods (see U.S. Pat. No. 7,695,752).

A recent, non-adhesive film method (mesodissection) and instrument (MilliSect) for tissue microdissection was has been developed which relies on physical removal of tumor cells from tumor tissue present in a tissue section via a milling device containing a milling blade that physically removes the tumor cells from the tissue section and collects them in the same milling device (see US Published Patent Application 2014/0329269).

The presently described signal directed tissue microdissection method relies on utilization of the DIRECTOR slide technology which provides many advantages over existing tissue microdissection methods. No previously described and/or developed methods are based on laser activation that is controlled by recognition of inclusion and exclusion signals encountered in a digital image of the tissue. The presence of both inclusion and exclusion signals provides a higher degree of precision by precisely identifying tumor, non-tumor boundaries. This is particularly advantageous since the laser beam can be as small as 1 μM in diameter which is approximately 1/10^{th} the diameter of a single cell. This means that when the activated laser beam approaches the heterogeneous boundary between tumor and non-tumor as identified by inclusion/exclusion signals, the beam is prevented from activating at the point of exclusion signal with a resolution at 1/10^{th} diameter of the cells thus very little chance of transferring non-tumor cells, or even a fraction of a non-tumor cell, from the tissue into the receiving vesicle.

Some of the other methods described above are based on contact of synthetic films with tissue whereby the film becomes activated by either a laser or simple white light such that activation of the film physically interacts with and “grabs” the cells of interest. The cells of interest can be identified for collection by IHC, in situ hybridization, and/or a trained histologist/pathologist utilizing staining patterns/morphological characteristics of cells and cell populations. Once film activation is completed the film which now “holds” the cells and cell populations of interest is placed into a receiving vesicle and the tissue is eluted from the film into a sample preparation buffer. These methods have the disadvantage of lack of precision due to possible contamination of non-tumor cells mixed in with desirable tumor cells whereby the non-targeted cells may inadvertently adhere to the film. This will result in the non-targeted cells making their way into the tube when the film is removed from the tissue and placed into the sample preparation tube making for a sample that contains the targeted tumor cells along with the non-targeted, non-tumor cells.

Another of the tissue microdissection methods described above which utilizes physical milling and removal of tissue from a tissue section also leads to a lack of precision based on physical limitations on the size of the milling needle. This method uses a needle that physically mills and removes the identified cells; however, the smallest diameter of a needle is 100 μM thus providing much less precision when attempting to cut out of a tissue section cells that are approximately 10 μM in diameter. The materials and process used for manufacturing the blade and collection device cannot physically make a blade that is down to the level of the diameter of a laser beam.

### SUMMARY

A tissue microdissection method is provided as part of a multiplex panomic data-generating process to inform optimal cancer treatment strategies. Because cancer therapeutic agents attack and kill tumor cells, and because these agents target specific proteins and/or cancer antigens, informing treatment decisions about which of these protein or antigenic targets to attack with which agents is critical and thus providing analysis of these target proteins and antigens in a pure population of tumor cells is the critical step in this multiplex panomic process. The result of this multiplex panomic process is assessing the proteomic and genomic status (panomics) of only the patient's tumor cells procured from the patient's tumor tissue utilizing the presently described direct signal tissue microdissection method. Once the panomic status of patient tumor cells has been determined, the most optimal treatment strategy for the cancer patient can be determined. The treatment strategy comprises biological, small molecule, chemotherapeutic, cancer vaccine, and immunomodulatory treatment agents, or some combination of such agents, which has been directly matched to the molecular characteristics of the patient's own tumor cells thus providing for a personalized strategy for cancer treatment. Various objects, features, aspects and advantages of the disclosed subject matter will become more apparent from the following detailed description of preferred embodiments, along with the accompanying drawing figures.

### BRIEF DESCRIPTION OF THE DRAWING

FIG. 1 shows a section of patient tumor tissue placed onto a DIRECTOR slide where inclusion and exclusion signals are assigned to specific cell populations either manually by a trained operator/histologist/pathologist or automatically by computer software, or a combination of both whereby the trained operator/histologist/pathologist utilizes a computer to aid in assigning inclusion/exclusion signals. These signals are assigned via strategies such as IHC, in situ hybridization, and/or visual histological evaluation by a trained operator/histologist/pathologist, in order to coordinate and orchestrate laser induced forward transfer of only those cells identified by an inclusion signal. The presence of the exclusion signal functions to improve identification of only the inclusion-specified cells, especially at margins of different cell populations, resulting in increased precision of laser activation resulting in higher purity of cell collection.

### DETAILED DESCRIPTION

The compositions and methods disclosed herein enable determination of the molecular status of a cancer patient's tumor cells thus informing the treatment of said patient with drugs and agents that are based solely on the molecular status of the tumor cells. This approach is the foundation of a personalized cancer therapy strategy. The molecular status of tumor cells precisely collected by the presently described method is performed by determining; 1) the genomic DNA sequence, 2) RNA expression, and 3) quantitative levels of specific drug target proteins and drug modulatory proteins. It is critical that the molecular status be determined on the tumor cells and only the tumor cells because cancer treatment strategies target killing of only the tumor cells. Thus precise collection of a pure homogeneous population of tumor cells from cancer patient tumor tissue is important so that the entirety of the molecular status reflects the tumor cells, and only the tumor cells. This presently described signal directed tissue microdissection method ensures that false information that would reflect the wrong type of cell being collected and analyzed from the patient tumor tissue will most likely not happen. It should be appreciated that the disclosed techniques provide fine grained control over boundary conditions between tumor and non-tumor regions in a tissue sample. Such control optimizes (e.g., maximizes) tumor capture while also optimizing (e.g., minimizing) non-tumor cells.

Detecting and determining quantitative levels of specific drug target proteins and drug modulatory proteins directly in tumor cells from patient tumor tissue is one aspect of the field of technology termed proteomics. Proteomics, and more specifically target proteomics, is performed by detecting and/or quantitating specified peptides derived from subsequences of full length proteins, such as cancer drug target proteins, using mass spectrometry-based Selected Reaction Monitoring (SRM), also referred to as Multiple Reaction Monitoring (MRM), and which is referred to herein as an SRM/MRM assay. The detection of mutations in the DNA present in patient tumor cells from patient tumor tissue is one aspect of the field of technology termed genomics, and is performed by detecting changes and/or variants from normal in the nucleic acid sequence from patient tumor cells collected using next generation sequencing (NGS) technology, whereby NGS technology is utilized to sequence an entire genome (whole genome sequencing [WGS]), sequence the entire collection of all the exons from all the genes present in a genome (whole exome sequencing [WES]), or sequence a pre-defined subset of the entire collection of exons from all the genes present in a genome (exome sequencing [ES]). Detection and quantitation of RNA expression in patient tumor cells collected from patient tumor tissue is yet another aspect of the technology field of genomics, and is performed by detecting and quantitating levels of all, or a subset of all, RNA molecules in patient tumor cells using such RNA analytical methods including but not limited to RNA-Seq (NGS), hybridization-based microarrays, RT-PCR, and quantitative RT-PCR. Combining both proteomics and genomics technologies to the analysis of patient tumor cells is termed panomics.

Detection and quantitation of specific proteins above or below specified quantitative levels in tumor cells procured from cancer patient tumor tissue using the presently described signal directed tissue microdissection method is used to indicate that a particular patient may be treated with a strategy that includes one or more therapeutic agents specifically designed to have an effect on the function of said proteins in order to negatively impact the growth of the tumor cells. If specific mutations are detected in the DNA of tumor cells procured from cancer patient tissue using the presently described signal directed tissue microdissection method, the patient may be treated with a regimen that includes one or more therapeutic agents designed to have a negative effect on the growth of the tumor cells where the therapeutic strategy is indicated by the detected mutation(s). Similarly, if expression of a specific RNA molecule, or collection of RNA molecules, is detected and/or specific levels quantitated and found above or below specified quantitative levels in tumor cells procured from cancer patient tumor tissue using the presently described signal directed tissue microdissection method, the patient may be treated with a regimen that includes one or more therapeutic agents designed to have a negative effect on the growth of the tumor cells as indicated by expression of a specific RNA molecule, or collection of RNA molecules. Finally, if neoantigens are present in patient tumor cells collected utilizing the presently described signal directed tissue microdissection method and which are not found in normal cells from the same patient and which are discovered using the described panomics approach, these neoantigens can be utilized therapeutically as tumor vaccines to modulate and elicit an immune response to the patient's own tumor cells. All of these described cancer treatment strategies are informed by use of the presently described signal directed tissue microdissection method that serves as the central component of this panomics multistep process for determining the molecular status of a patient's tumor cells.

The presently described signal directed tissue microdissection method provides the ability to precisely procure previously-specified populations of cells from a tissue section containing a heterogeneous mixture of cells whereby the specific cell populations for collection are pre-identified by an inclusion signal and where a pre-identified exclusion signal is used to prevent collecting unwanted populations of cells from the same heterogeneous tissue.

Tissue staining methods used by a trained histologist/pathologist to visualize specific cells and cell populations in tissue sections to guide the marking of cells and cell populations with inclusion/exclusion signals can take the form of colorimetric stain, chemically-induced stain, or fluorescent stain. There are many standard histology stains that result in a wide range of visual colors that are well established and well known in the field of histology/pathology and include but are not limited to hematoxylin, eosin, congo red, aldehyde fuchsin, anthraquinone derivatives, alkaline phosphatase, Bielschowsky, cajal, cresyl violet, Fontana-Masson, Giemsa, golgi stain, iron hematoxylin, luxol fast blue, luna, Mallory trichrome, Masson trichrome, Movat's pentachrome, mucicarmine, nuclear fast red, oil red O, orcien, osmium tetroxide, Papanicolaou, periodic acid-schiff, phosphotungstic acid-hematoxylin, picrosirius red, Prussian blue, reticular fiber, Romanowsky stains, safranin O, silver, sudan stains, tartrazine, toluidine blue, Van Gieson, Verhoeff, Von Kossa, and Wright's stain. Each type of stain generates different colors to the human eye and/or spectrum channels in a digital image. Thus, one aspect of the disclosed subject matter is mapping the color (e.g., RGB, HSV, etc.) or spectrum (e.g., wavelength of light, etc.) channels to inclusion or exclusion signals.

There are many methods for developing a chemically-induced visual color in tissue sections in order to guide the marking of cells and cell populations with inclusion/exclusion signals for the presently described method including but not limited to immunohistochemistry and in situ RNA hybridization. Immunohistochemistry (IHC) determines in what region and in which cells of the tissue a specific protein and/or peptide is expressed. IHC relies on the binding of a primary antibody that has been developed to have specific binding properties to a particular target protein and/or peptide whereby a tissue section on a slide is incubated with the primary antibody so that the primary antibody binds to its target protein and/or peptide as it resides within the regions and cells of the tissue. Once this binding reaction has occurred, the tissue section is then incubated in the presence of a secondary antibody that specifically binds the primary antibody. The secondary antibody has been engineered with a specific molecule that has the ability to elicit a chemical reaction based on treatment with other chemicals to render a specific color. The tissue is then incubated under various biochemical conditions with specific other chemical reagents designed to impart the specific color to the secondary antibody. Thus where the secondary antibody binds its primary antibody target that is where the colorimetric stain will present. A similar approach is used to develop a colorimetric stain in the process of in situ hybridization with nucleic acid probe/hybridization methodology. A specific nucleic acid that is 100% complementary to a target RNA molecule is used to determine which regions and in which cells of the tissue a specific target RNA molecule is expressed in a tissue section. A tissue section is incubated under experimental conditions whereby a complementary nucleic acid probe binds to the target RNA molecule. The nucleic acid probe was previously modified so that when incubated with a specific chemical reagent a colorimetric stain will result. Once the probe has been bound to the tissue section the section is then incubated with specific chemical reagents designed to elicit the colorimetric stain and in this way only those regions and cells of the tissue where the target RNA molecule is present will have a colorimetric stain.

Chemical reagents that induce colorimetric stains in an immunohistochemical and/or in situ RNA hybridization assay include but are not limited to 3,3′-Diaminobenzidine, 5-Bromo-4-chloro-3-indolyi phosphate, methyl green, PTAH, toluidine blue, PAS, luxol fast blue, and Wright's stain. There are also many ways in which to develop a fluorescent stain using fluorescent signal emission molecules to impart a specific color to an immunohistochemical and/or in situ RNA hybridization assay including but not limited to fluorescein, carboxyfluorescein, rhodamine, coumarin, and cyanine. These lists are not meant to be all inclusive but simply by way of example. Those trained in the art will recognize all various methods of developing a colorimetric stain using standard histology, immunohistochemical, and/or in situ RNA hybridization methodology that can serve to ascribe either an inclusion signal or an exclusion signal to specified cells and cell populations within tissue within the presently described method.

The most effective way of deciding which cells and cell populations should be identified with inclusion and exclusion signals is done by a trained histologist/pathologist whereby he/she visualizes colors imparted to the tissue and cells by the methods previously described including but not limited to standard histological stains and/or IHC/in situ hybridization. Various colors are imparted to the tissue and cells using these methods whereby tumor cells will comprise a different staining color than benign, non-tumor cells such as stroma and lymphocytes. In addition, tumor cells show very different physical and morphological features than benign, non-tumor cells such as stroma and lymphocytes. Thus the trained histologist/pathologist will utilize a combination of knowledge comprising unique staining colors and unique morphological features of tumor cells and benign non-tumor cells to identify and impart inclusion signals to the tumor cells and exclusion signals to the benign non-tumor cells.

In one iteration the selection process of ascribing inclusion and/or exclusion signals to cells and cell populations within a tissue section on a DIRECTOR slide is based on the standard methodology of human recognition of cell staining, cell staining patterns, and histological features of specific cells within a heterogeneous tissue section by a trained histologist/pathologist. In this process of ascribing inclusion/exclusion signals to a tissue section, the trained histologist/pathologist uses a computer, computer software, and a computer monitor to interact with; 1) a live image of the tissue on a microscope that interacts with said computer, or 2) a digital image previously imaged on a microscope scanner and displayed on said computer monitor. The trained histologist/pathologist then digitally marks inclusion/exclusion signals on the virtual image of the tissue section using the mouse of the computer, or a stylus on a touch screen display monitor that is displaying either the live image or a digital image of the tissue section. Imparting these signals to tumor cells and benign non-tumor cells on either a live image or a digital image of the stained tissue is performed by the trained histologist/pathologist via visualizing the cellular stains and cellular morphological differences through a microscope or on a digital image of the tissue developed on a slide scanner after staining. The trained histologist/pathologist will then interact with a computer screen displaying the image whereby he/she will utilize either the mouse of the computer or a stylus on a touch screen monitor to mark cells with inclusion/exclusion signals via software that allows for the ability to digitally mark a digital image or a live image with this additional information.

Once specific cells/cell populations have been identified and digitally marked on the digital or live image of a tissue section by the trained histologist/pathologist, a digitally imprinted image is prepared in a standard digital imaging format such as for example PNG, TIFF, GIF, JPEG, PDF, SVS, or other digital format. The image comprises digitally imprinted cells identified by specific inclusion/exclusion signals within patient tumor tissue whereby such specified cells should or should not be collected and which such information is utilized by the computer in order to control laser activation.

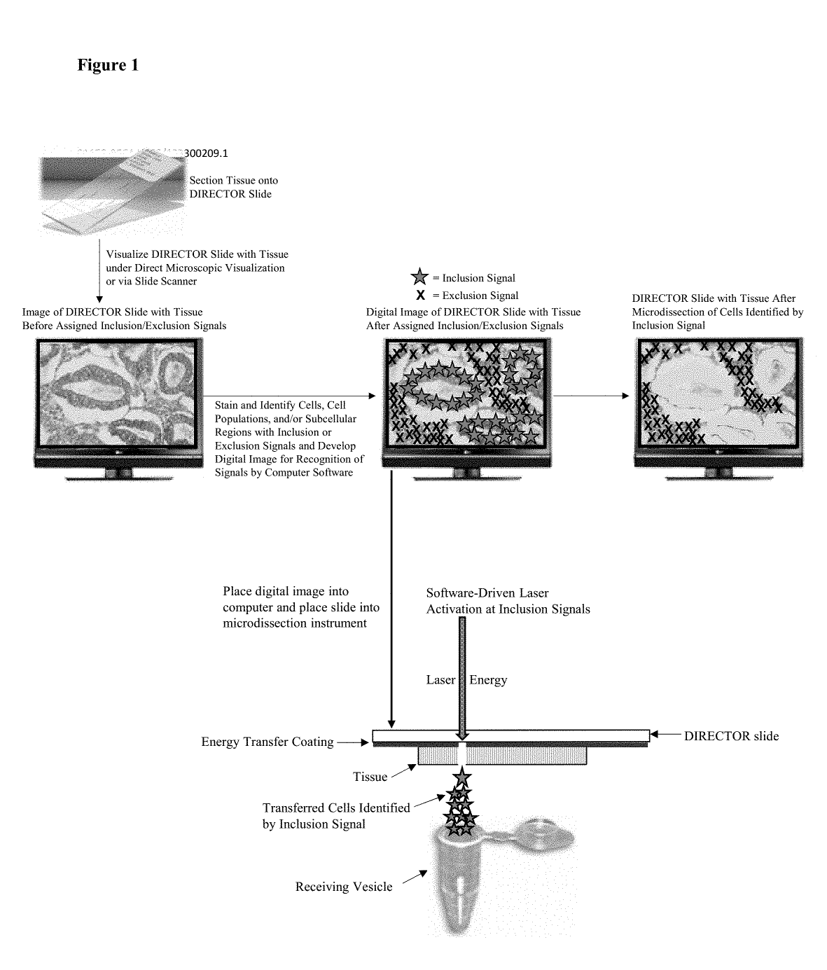

The presently described signal directed tissue microdissection method is graphically depicted in FIG. 1 and demonstrates how this method is based on an instrument platform. Such an instrument comprises at least the following parts and operational functions: 1) a moveable microscope slide stage to hold and immobilize a DIRECTOR slide, or slides, that will precisely move, as designated by the inclusion and exclusion signals, in relation to a fixed laser source, 2) a computer monitor to display a live image of the tissue section to be microdissected as visualized through either a digital camera or microscope objective that magnifies an image of the tissue section through a digital camera, 3) a computer and computer software that digitally compares and precisely aligns the virtual imprinted digital image of the tissue section containing the inclusion/exclusion signals with the live image of the exact same tissue section on the DIRECTOR slide as it resides within the moveable microscope slide stage, 4) a laser (as for example a solid state ND:YAG at wavelength of 355 nm) that is capable of emitting a laser beam of approximately 1-50 μM in diameter and which can effectively strike and vaporize the energy transfer coating of the DIRECTIR slide, 5) a computer and computer software that activates the laser to strike the energy transfer coating of the DIRECTOR slide at only those cells and cell populations identified by the inclusion signal and which reside on the energy transfer coating of the DIRECTOR slide opposite to the laser beam, 6) the ability to collect and accumulate the laser-induced forwardly transferred cells and cell populations from the DIRECTOR slide.

DIRECTOR slides comprise a standard glass slide that has been coated with an optically-translucent energy transfer layer that allows for Laser Induced Forward Transfer (LIFT) technology. LIFT technology is defined as the movement of objects with laser energy via a thin energy transfer layer. A tissue section is placed on a DIRECTOR slide and standard histological methods are utilized to prepare the tissue for histological analysis and tissue microdissection. Once cells of interest are identified a pulsed photon laser energy contacts the energy transfer coating resulting in an explosive event that instantly transfers the cells of interest into the collection tube below via a heat-induced explosive event. The energy transfer coating absorbs all of the laser energy, so the biomolecules in the sample are not affected. In addition, the layer is completely vaporized thus there is absolutely no contamination of the dissected tissue. The method describing the use of a DIRECTOR slide for laser induced forward transfer of tissue via utilization of an energy transfer interlayer coating is described in U.S. Pat. No. 7,381,440, the contents of which are hereby incorporated by reference in their entirety.

The tumor cells are the target of cancer drug therapeutic agents thus this signal directed tissue microdissection provides for a pure homogeneous collection of patient tumor cells for informing the cancer treatment decision about which therapeutic agent or agents should be used to treat the patient. The presently described signal directed tissue microdissection method is the cornerstone for a multistep panomics process comprising: 1) obtaining formalin fixed paraffin embedded tumor tissue via a physician and/or healthcare team accompanied by a test requisition form describing the requested tests, 2) isolating and collecting a purified population of patient tumor cells directly from said patient tumor tissue using the presently described signal directed tissue microdissection method, 3) reducing said population of patient tumor cells to a soluble and liquefied state using standard tissue sample preparation protocols and reagents and/or the Liquid Tissue protocol and reagents, 4) detecting and quantifying targeted proteins in said Liquid Tissue lysate using mass spectrometry to develop protein expression profiles and hence the proteomic status of the patient's tumor cells, 5) determining the genomic status of the patient's tumor cells by detecting mutations in DNA and RNA present in said Liquid Tissue lysate, and/or another lysate prepared from microdissected tumor cells by such methods as nucleic acid sequencing, next generation sequencing, microarray assay, RNA-seq, PCR, RRT-PCR, and/or Q-RT-PCR, 6) detecting RNA expression profiles in the lysates prepared from microdissected tumor cells by such methods as nucleic acid sequencing, RNA-seq, PCR, RRT-PCR, microarray assay, and/or Q-RT-PCR and hence a further genomic status of the patient's tumor cells, 7) informing an optimal cancer treatment strategy from which the patient will most likely benefit wherein said strategy is based on the combination of the protein expression status, DNA mutation status, and RNA expression status obtained from said patient's tumor cells that were collected using the presently described signal directed tissue microdissection method, 8) preparing a patient report containing all the protein expression, DNA mutation, and RNA mutation/expression information about the patient's tumor cells in order to convey said scientific data and optimal treatment strategy about said patient's tumor cells to the cancer patient's medical professional team including said patient's physician.

Once a homogeneous collection of tumor cells is isolated and collected via the presently described signal directed tissue microdissection method, the cells can be liquefied into a complex multi-use biomolecule lysate. One method of preparing complex biomolecule samples directly from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocol available from Expression Pathology Inc. (Rockville, Md.).

The most widely and advantageously available form of tissue, including tumor tissue, from cancer patients is formalin fixed, paraffin embedded tissue (FFPE). Formaldehyde/formalin fixation of surgically removed tissue is by far and away the most common method of preserving cancer tissue samples worldwide and is the accepted convention in standard pathology practice. Aqueous solutions of formaldehyde are referred to as formalin. “100%” formalin comprises a saturated solution of formaldehyde (this is about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods that can analyze formalin fixed cancer tissue is considered suitable and heavily utilized methods for analysis of cancer patient tissue. The presently described tissue microdissection method is particularly useful for collecting specific cell populations from FFPE tissue; however, this presently described method will also microdissect tissue that has been frozen.

It should be noted that any language directed to a computer should be read to include any suitable combination of computing devices, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed signal directed tissue microdissection method. Further, much of the disclosed method can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

As used in the description herein and throughout the claims that follow, when a system, engine, server, device, module, or other computing element is described as configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

### Description of FIG. 1

The presently described signal directed tissue microdissection method is depicted in FIG. 1. Tissue is sectioned onto a DIRECTOR slide and stained using but not limited to standard histological stains, IHC, and/or in situ hybridization. Based on staining patterns and tissue/cell morphological characteristics revealed by the staining, inclusion and exclusion signals are determined automatically using a computer and computer software, by a trained histologist/pathologist, or a combination of both. A digital image of the tissue containing inclusion/exclusion signals is developed in standard digital imaging format and loaded into a computer that drives an instrument comprising a laser, a moveable microscope slide stage, and a receiving vesicle. A computer and computer software is used to activate the laser only at points where inclusion signal is encountered and the laser is prevented from activating at points where exclusion signals are encountered. Thus only those cells, cell populations, and/or subcellular regions identified by inclusion signal are transferred downward into the receiving vesicle.

### Description of Multistep Panomic Process

Patient tumor tissue is used for the presently described signal directed tissue microdissection method which is the focal point of a multistep panomic approach to informing optimal cancer therapeutic approaches. This process begins with acquisition of a patient's formalin fixed paraffin embedded (FFPE) tumor tissue sample through the patient's attending physician and/or professional healthcare team. This team can include but is not limited to the patient's primary care physician, oncologist, molecular oncologist, clinical nurse, pathologist, molecular pathologist, radiologist, surgeon, physician's assistant, and/or additional consulting physicians. In this process, the primary contact and healthcare professional in possession of the patient tumor tissue prepares a clinical requisition form and the tissue is sent accompanied by the clinical requisition form to the CLIA-certified clinical laboratory that performs this process. The tissue can be sent as a whole FFPE tissue block whereby the tissue will be sectioned onto DIRECTOR slides in the CLIA-certified clinical laboratory that performs this process or tissue can be sent in the form of tissue sections previously cut onto DIRECTOR slides for tissue microdissection to be performed directly from those slides.

The clinical requisition form includes but is not limited to the following HIPPA-compliant information: 1) the ordering physician information, 2) information about the pathology laboratory sending the tissue including name and address, 3) information about the specimen itself including the type of tissue, the type of cancer, and the organ or origin, 4) patient information including name, address, date of birth, and medical record number, 5) private insurance and Medicare/Medicaid information for billing purposes, 6) hospital discharge date, and 7) which test or tests the attending physician is ordering.

Once the CLIA-certified clinical laboratory is in possession of the tissue on DIRECTOR slides, the next step is to collect tumors cells for analysis using the presently described signal directed tissue microdissection method. Patient tumor tissue is highly heterogeneous in its cellular makeup comprising a variety of different types of cells including among others tumor cells, normal structural fibroblasts, infiltrating lymphocytes, and normal epithelial cells. The tumor cells in surgically removed patient tumor tissue are thus intermixed with different types of cells that are not tumorigenic and which are not the cellular targets of cancer therapeutic strategies. Thus if whole tumor tissue were to be used to make a soluble, liquefied biomolecule lysate for molecular analysis the resulting information would not be specific to only the tumor cells which are the targets of cancer therapeutic strategies. The goal is for cancer therapeutic strategies is to attack only tumor cells thus it becomes paramount that all molecular data important to informing a decision about which cancer therapeutic strategy will likely be the most effective at killing the tumor cells must be specific to only the tumor cells and not normal cells. This fact makes the presently described signal directed tissue microdissection method the most critical component in this multistep panomics process for informing optimal cancer therapy treatment decisions.

One part of this multistep panomics approach to informing optimal cancer treatment decisions is determining quantitative expression of specific drug target proteins in tumor cells collected from patient tumor tissue using the presently described signal directed tissue microdissection method. This is performed by a mass spectrometer using the SRM/MRM method, whereby the SRM/MRM signature chromatographic peak area of each peptide is determined within a complex peptide mixture present in a Liquid Tissue lysate (see U.S. Pat. No. 7,473,532, as described above). Quantitative levels of cancer drug target proteins are determined by the SRM/MRM methodology whereby the SRM/MRM signature chromatographic peak area of an individual specified peptide from each of the cancer drug target proteins in one biological sample is compared to the SRM/MRM signature chromatographic peak area of a known amount of a “spiked” internal standard for each of the individual specified cancer drug target protein fragment peptides. Software specifically developed for analysis of SRM/MRM data is utilized to quantitate each and every protein that has been assayed by the SRM/MRM assay. Because SRM/MRM assays are very sensitive it is imperative that non-tumor cells be excluded from analysis of patient tumor tissue, and the presently described method imparts an exclusion signal to prevent benign, non-malignant cells such as normal cells from contaminating analysis of tumor cells, which creates an optimized sample. Changes in quantitative levels of specific oncoproteins, and/or peptides from proteins, in patient tumor cells can inform the cancer treatment decision whereby elevated levels of proteins, and/or peptides, are identified as potential therapeutic targets because they can drive the growth of the tumor cells, mask the tumor cells from the patient's own immune surveillance system, or provide for cancer vaccine targets to arm the patient's own immune system against the tumor cells. Changes in qualitative characteristics of specific oncoproteins, and/or peptides from proteins, in patient tumor cells can also inform the cancer treatment decision whereby proteins, and/or peptides, that derive from mutated DNA can also be potential therapeutic targets because they can drive the growth of the tumor cells, mask the tumor cells from the patient's own immune surveillance system, or provide for cancer vaccine targets to arm the patient's own immune system against the tumor cells.

SRM/MRM assays are capable of not only quantifying proteins and peptides but are also capable of determining the amino acid sequence of specific proteins and peptides, and thus determining if a specific protein and/or peptide found to be expressed in tumor cells microdissected using the presently described signal directed tissue microdissection results from expression of a specific mutated form of a stretch of DNA in the tumor cells. This approach to determining the amino acid sequence of specific proteins and peptides provides relevant quantitative proteomics expression level for genes and can determine if candidate neoantigens identified by genomics methods are translated into proteins/peptides that may reside on the surface of patient tumor cells. Thus both quantitative and qualitative proteomics data garnered from such SRM/MRM assays becomes a focal point for informing about the use of a number of therapy approaches including but not limited to biological agents, small molecules, chemotherapeutic agents, cancer vaccines, and immunomodulatory agents.

Another part of this multistep panomics approach to inform optimal cancer treatment decisions is mutation analysis of DNA in tumor cells collected from microdissected patient tumor tissue using the presently described signal directed tissue microdissection method. This is performed through whole genome sequencing (WGS), whole exome sequencing (WES), or subsets of whole exomes primarily through the methods of DNA sequencing including but not limited to Next Generation Sequencing (NGS) methods. DNA is prepared from cells collected using the presently described signal directed tissue microdissection. DNA preparation methods or using the Liquid Tissue® lysate. The goal of WGS is to sequence each and every nucleic acid base, including but not limited to all the introns, exons, intervening sequences, repeats, or other features present in the tumor cells collected by the presently described signal directed tissue microdissection method. The definition of mutation in the DNA of tumor cells procured from patient tumor tissue includes but is not limited to single nucleotide changes, insertions, deletions, rearrangements, duplications, duplications/deletions of individual nucleotides, duplications/deletions of multiple nucleotides, single base pair polymorphisms, transitions, transversions, inversions, copy number variations, duplications/deletions of long stretches of nucleic acids, or combinations thereof.

One skilled in the art will recognize the wide breadth of changes to the genome that constitute and characterize a mutation. Alternatively, less complex subsets of the genome can be targeted for sequencing to detect mutations by performing NGS of only exons, also termed exomes, including but not limited to whole exome sequencing (WES) and/or targeted exome sequencing (ES). Multiple mutations to inform optimal cancer therapy can be detected through WGS, WES, and ES as for example KRAS, BRAF, EGFR, and HER2 mutations. In addition, copy number variations and gene rearrangements as for example Her2, ALK, Met, and TOPO2A genes can be assessed by WGS through NGS methodology. Because NGS is very sensitive it is imperative that non-tumor cells be excluded from analyses performed as a product of this multistep panomics process, and the presently described signal directed tissue microdissection method imparts an exclusion signal to prevent normal cells from contaminating analysis of tumor cells.

Changes in DNA from the normal cell to the tumor cell can inform the cancer treatment decision whereby when a change in the DNA results in a change in the amino acid sequence of the resulting protein then this oncogenic protein, and/or the peptide that contains the changed amino acid, can become a focal point for killing the tumor cells using a number of therapy approaches including but not limited to biological agents, small molecules, chemotherapeutic agents, cancer vaccines, and immunomodulatory agents. Changes in the sequences of proteins and peptides as evidenced by changes in the DNA sequence of patient tumor cells can inform the cancer treatment decision whereby the specific proteins, and/or peptides, that result from tumor-specific changes in the DNA are identified as potential therapeutic targets because they can drive the growth of the tumor cells, mask the tumor cells from the patient's own immune surveillance system, or provide for cancer vaccines to arm the patient's own immune system against the tumor cells. Thus it is imperative that whole genome sequencing of normal blood cells obtained from the patient be performed in order to know the normal baseline genomic status of the patient so that changes from the normal that are found in the tumor cells can be attributed to the tumor cells.

Still another part of this multistep panomics approach to informing optimal cancer treatment decisions is the sequencing of RNA molecules and analysis of RNA expression performed by the methods of RNA-seq using the NGS platform, reverse transcription polymerase chain reaction (RT-PCR), and quantitative reverse transcription polymerase chain reaction (Q-RT-PCR). The RNA-seq method is performed using RNA prepared from the patient tumor cells in order to detect expression and determine the sequence of RNA molecules in tumor cells collected from patient tumor tissue using the presently described signal directed tissue microdissection method. This provides relevant RNA expression level for genes, transcribed neoantigenic RNA molecules, and/or non-transcribed RNA molecules. RNA-seq can be performed on the entire complement of all RNA molecules expressed in said tumor cells or RNA-seq can be performed on a subset of all expressed exons. RNA expression analysis in tumor cells can also be performed utilizing microarray technology and RT-PCR/Q-RT-PCR, both of which are also capable of providing relevant RNA expression as well as the sequence of all or specific populations of RNA molecules, transcribed neoantigenic RNA molecules, and/or non-transcribed RNA molecules. One skilled in the art will recognize that multiple methods can be employed to detect expression and determine the sequence of RNA molecules in a biochemical lysate containing RNA and that the methods described here are by example only and not meant to encompass all current and/or future methods to detect RNA expression levels and the sequence of the RNA molecules. RNA for RNA-seq can be prepared using RNA preparation methods or using the Liquid Tissue® protocol and reagents from tumor cells collected using the presently described signal directed tissue microdissected method. Changes in the sequences of RNA molecules as evidenced by changes in the RNA sequences found in patient tumor cells can inform the cancer treatment decision whereby the specific proteins, and/or peptides, that result from translation of tumor-specific RNA molecules are potential therapeutic targets because they can drive the growth of the tumor cells, mask the tumor cells from the patient's own immune surveillance system, or identify tumor-specific neoantigens to provide for cancer vaccines to arm the patient's own immune system against the tumor cells.

Results from target protein expression assays using SRM/MRM, mutation analysis of DNA using NGS, and expression analysis of RNA using NGS, RT-PCR and/or Q-RT-PCR can be used to correlate accurate and precise quantitative protein levels, detect mutations in genes, determine RNA expression patterns, and determine if mutations detected in tumor cell DNA are transcribed into RNA and translated into peptides and proteins. In order for this multistep process to be effective this information must be specific to only the tumor cells procured from cancer patient tissue, and this specificity is a direct result from using the presently described signal directed tissue microdissection method. These data are then used to match the most effective treatment options to the proteins and peptides that are aberrantly expressed specifically in the tumor cells. Many proteins and peptides that are aberrantly expressed in tumor cells and that drive the tumor cells to grow and divide have been targeted by the pharmaceutical industry whereby synthetic chemical molecules and/or biological molecules have been developed to specifically inhibit the function of these targeted proteins and peptides. These chemical and/or biological molecules that inhibit the function of these proteins and peptides are the cancer therapeutic agents and strategies used as cancer treatment regimens, whereby the treatment regimen will inhibit the function of these proteins and peptides and thus inhibit growth of the tumor cells.

Expression of specific proteins on the cell surface of tumor cells that function to mask the tumor cells from the patient's own immune surveillance system have also been the focus of targeted therapy approaches in recent years. A number of biological molecules and small molecules have been developed by the pharmaceutical industry to interact with these types of masking proteins that reside on the cell surface of tumor cells whereby these new cancer therapy molecules function to unmask the tumor cells and allow the patient's own immune system to identify the tumor cells as foreign to the body which in turn can help to mount a tumor-killing immune response to the tumor cells. Expression of these immune system-masking proteins are effectively analyzed by the described multistep process using the SRM/MRM methodology and wherein the presently described signal directed tissue microdissection method is the foundation of this multistep process through precise collection of only tumor cells from patient tumor tissue.

In addition to the cancer therapeutic agents that specifically kill tumor cells by inhibiting the function of oncoproteins that are driving tumor cell growth, the presence of neoantigens on the cell surface of tumor cells can also be exploited to act as a patient-specific vaccine targets that can be exploited to elicit an immune response by the patient to attack and kill his/her own tumor cells. Patient-specific cancer antigens (neoantigens) that reside on the surface of tumor cells and not on the surface of normal cells in the same patient can be used as cancer vaccine targets for use in arming the patient's own immune system to attack and kill the tumor cells. The discovery and analysis of tumor-specific antigenic proteins and peptides (neoantigens) is effectively performed by the described multistep process and wherein the presently described signal directed tissue microdissection method is the foundation of this multistep process through precise collection of only tumor cells from patient tumor tissue. Such patient-specific neoantigens can be identified by whole genome sequencing of the tumor cell DNA using NGS and comparing to the whole genome sequence of normal cells from the same patient, which are usually cells from the blood. Mutations in regions of transcribed DNA that change the amino acid sequence of the translated protein or peptide as identified by this approach of comparing tumor cell DNA sequence to normal blood cell DNA from the same patient can be considered potential neoantigens. Additional sequence analysis from the whole genome sequence of the tumor cell DNA can also reveal if these candidate neoantigens will appear on the cell surface of the tumor cells. The method of RNA-seq can be used to determine if these candidate neoantigens are expressed in the form of RNA suggesting that any RNA molecule found to represent a candidate neoantigen is translated into peptide and/or protein. The SRM/MRM method as applied to the tumor cells as previously described can also indicate if these candidate neoantigens are present on the tumor cell surface which would instantly identify any candidate neoantigen as a validated cancer vaccine target. This multistep process identifies these neoantigens on an individual patient basis and this ability is a direct result of providing a panomic tumor cell analysis approach centered on the presently described signal directed tissue microdissection method.

The described multistep process optimally informs a cancer therapeutic strategy, or combination of strategies, that would most likely be successful in inhibiting tumor cell growth in a cancer patient and helps direct a physician and/or other medical professional to determine appropriate therapy for the patient by matching the available cancer therapeutic agent, or agents, with those cancer proteins or peptides that are found to be aberrantly expressed in the tumor cells of the patient. Having knowledge of both the proteomic and genomic status of a patient's tumor cells, utilizing this multistep process, informs the individualized cancer treatment strategy for that given patient. Any given cancer treatment strategy targets specific proteins and/or specific peptides present in the tumor cells whereby the most optimal biological, small molecule, chemotherapeutic, cancer vaccine, and immunomodulatory treatment strategy is directly matched to the molecular characteristics of the patient's own tumor cells identified by the combined proteomic and genomic assays. Biological agents, small molecules, and standard chemotherapeutic agents function by binding to specific, targeted proteins to inhibit their function and/or aid in eliciting a patient immune response to the tumor. Immunomodulatory agents and cancer vaccines function to elicit an immune response from the patient to the tumor cells. Treating a patient with more than one of these treatment strategies is more effective in general than treating with only one of these strategies because tumor cells frequently express one or more such target proteins and/or peptides simultaneously. This is because it is much more difficult for a tumor cell to evade and develop resistance to multiple agents at once. Thus it is particularly advantageous to design treatment strategies based on combining one or more treatment strategies comprising a biological agent such as cetuximab, a small molecule biochemical agent such as lapatinib, a standard chemotherapeutic agent such as gemcitabine, an immunomodulatory agent such as nivolumab, and/or a cancer vaccine agent such as a protein/peptide identified as a candidate neoantigen from patient tumor cells. The presently described signal directed tissue microdissection method to precisely collect only tumor cells from patient tumor tissue is the central focal point of this multistep panomics approach to deciphering the genomic/proteomic makeup of the patient's own tumor cells in order to determine which proteins/peptides to target with treatment strategies and agents. In this way this multistep process functions to derive the most optimal personalized cancer therapy for that patient, either with single agent treatment strategies or a strategy that includes multiple agents in combination.

Treatment strategies can be on an individual agent basis or a combination of multiple agents, all based on determining the proteomic/genomic status of the patient tumor cells using the described multistep process which relies on the presently described signal directed tissue microdissection method. The following 5 scenarios highlight how this approach might inform personalized cancer treatment strategies.

Scenario 1: If a patient's tumor cells are discovered by SRM/MRM methodology to express high levels of the Her2 protein and high levels of the PDL1 protein then a logical treatment strategy is to treat the patient with trastuzumab which attacks and inhibits the growth of cells expressing the Her2 protein in combination with nivolumab which masks the PDL1 protein to allow the immune system to recognize the tumor cells as foreign. This strategy would hopefully result in the patient mounting an effective immune killing response to the tumor cells in combination with inhibition of tumor cell growth due to treatment with trastuzumab.

Scenario 2: If a patient's tumor cells by SRM/MRM methodology are found to express high levels of the EGFR protein and whole genome DNA sequencing and RNA-seq methodology identifies expression of a candidate neoantigen then a treatment strategy would be to treat the patient with cetuzimab to inhibit growth of the tumor cells in combination with a patient-specific cancer vaccine to arm the immune system to attack the tumor cells that express the newly discovered neoantigen. In this way targeted tumor cell killing and a patient-mounted immune response would be combined to kill tumor cells.

Scenario 3: If a patient's tumor cells are discovered by SRM/MRM methodology to express high levels of the FR-α protein, low levels of the GART protein, and high levels of the EGFR protein then a treatment strategy would be to treat the patient with the chemotherapy agent pemetrexed in combination with cetuximab. The function of pemetrexed is to inhibit the biochemical function of GART, TS, and DHFR proteins. FR-α mediates active uptake of pemetrexed into the tumor cells and thus the higher the levels of FR-α the more pemetrexed gets into the cell resulting in the tumor cells being unable to synthesize nucleic acids preventing cell division and ultimately killing the tumor cells. The function of cetuximab is to bind to the EGFR protein and inhibit the growth of the tumor cells.

Scenario 4: If a patient's tumor cells are discovered by SRM/MRM methodology to express high levels of the FR-α protein and low levels of the GART protein, and are discovered by whole genome sequencing and RNA-seq methodology to express a candidate neoantigen then a treatment strategy would be to treat the patient with pemetrexed in combination with a patient-specific vaccine to arm the immune system to attack the tumor cells that express the newly discovered neoantigen. In this way targeted tumor cell killing and a patient-mounted immune response would be combined to kill tumor cells.

Scenario 5: If a patient's tumor cells are discovered by SRM/MRM methodology to express high levels of the PDL1 protein and high levels of the TOPO2 protein, while a candidate neoantigen is identified by DNA sequencing and RNA-seq then a treatment strategy might combine 3 different treatment agents. The first is to treat the patient with nivolumab which masks the PDL1 protein to allow the immune system to recognize the tumor cells as foreign. The second in combination with nivolumab is to treat with the chemotherapeutic agent doxorubicin which inhibits the normal function of TOPO2 protein to prevent DNA damage repair in the tumor cells. The third agent in combination with the nivolumab and doxorubicin is to treat with a patient-specific cancer vaccine that targets the newly discovered candidate neoantigen. This strategy would hopefully result in the patient mounting an effective immune killing response to the tumor cells in combination with inhibition of tumor cell growth due to treatment with doxorubicin.

It is clear from these 5 hypothetical scenarios that informing an optimal cancer treatment strategy from which the patient will most likely benefit is advantageously based on the combination of the protein expression status, DNA mutation status, and RNA expression status obtained from said patient's tumor cells that were collected using the presently described signal directed tissue microdissection method.

An important aspect of this multistep panomic process that is based on the presently described signal directed tissue microdissection method is the development and delivery of an individualized patient report that is sent from the CLIA-certified clinical laboratory that performed the claimed process to the patient's attending physician and/or healthcare team. This individualized patient report contains the proteomic and genomic status of the tumor cells resulting from the multistep molecular analysis process and includes the following: 1) HIPPA-compliant/physician-relevant information about the patient including but not limited to name, gender, date of birth, medical record number, incoming specimen number, testing laboratory number, requisition number, date specimen was received, date the report was sent, referring physician, physician institute, and the pathology institute, 2) a digital histological image of the specimen, 3) pathology/histology information about the specimen including but not limited to the diagnosis code, the specimen source, pathologist comment about the histology and adequacy for tissue microdissection and proteomic/genomic analysis, 4) results highlight summarizing the critical proteomic/genomic findings, 5) the proteomics results across all proteins analyzed, 6) the genomics results across all nucleic acids analyzed, 7) the clinical implications of each significant proteomic and genomic finding with respect to which therapeutic agents and/or therapeutic strategy that may likely provide the greatest potential benefit to the patient, which therapeutic agents and strategy would show uncertain benefit for the patient, and which therapeutic agents and strategy would likely provide a reduced likelihood of benefit to the patient, 8) assessment of the histological characteristics of the tumor based on proteomic and genomic analysis, 9) review and signature of the Medical Director of the CLIA-certified clinical laboratory.

The following example is a real-world experience resulting from use of the described multistep panomic process to inform the cancer therapy decision for this particular patient.

### EXAMPLE

### Determination of an Optimal Cancer Treatment Regimen Utilizing the Claimed Process

### Patient

### Female, Age 44

Patient presented with poorly differentiated cervical cancer. There was no recent history of abnormal pap smears. A radical hysterectomy with ovary preservation was performed. Pathology analysis showed localized wall invasion to the outer 3^{rd} of the cervix with no lymph node involvement. First line chemotherapy was platinum-based regimens for 3 months at which point disease recurrence was found by CT/PET scan. Patient developed renal failure and declined further treatment. The attending physician decided to utilize the claimed process to investigate the potential for identifying a therapeutic agent from which this patient may achieve some beneficial treatment.

### Method

The surgically-removed FFPE tumor tissue was received in the CLIA-certified clinical diagnostic laboratory, along with the requisition form, and analyzed using the claimed process. Tumor cells from FFPE patient tumor tissue were identified by a clinically-trained pathologist and collected using DIRECTOR-based tissue microdissection. Tumor cells were liquefied using the Liquid Tissue protocol and reagents for downstream proteomic and genomic analysis. Protein levels were quantitated by SRM assays and gene mutations detected by whole genome sequencing (WGS). A patient report was prepared and sent to the patient's attending physician containing this information as well as a suggested treatment regimen based on this information.

### Result

Quantitative SRM/MRM data for 27 proteins indicates that the Her2 protein was abnormally highly expressed. The other proteins for which inhibitory therapeutic agents are available were found to not be highly expressed. WGS indicated that the Human Papilloma Virus (HPV) had inserted into the DNA of the tumor cells directly adjacent to the Her2 gene, presumably causing amplification of the Her2 gene. This may likely be the reason for high expression of the Her2 protein. There were no additional findings in the sequencing information that could provide insight into additional therapeutic choices.

### Treatment Decision and Patient Outcome

After receiving the patient report showing these results and armed with this important information, the attending physician treated the patient with multiple inhibitors of the Her2 protein in order to stop growth of the tumor cells including the combination of trastuzumab and lapatinib. After 2 months of treatment, a CT/PET scan revealed significant tumor shrinkage. Based on tumor progression 3 months later, the patient was switched to a new combination of Her2 inhibitor therapy using the agents T-DM1/pertuzumab/lapatinib and subsequent analysis showed tumor regression.

### Conclusion

Overexpression of the Her2 protein and Her2 gene amplification are not common occurrences in cervical cancer, and has not been previously described in the scientific literature, so routine Her2 testing for protein expression and Her2 gene amplification is not performed for cervical cancer. At the last follow up examination, this patient has been on Her2 protein inhibitor therapy for 9 months. The median survival of all recurrent cervical cancer patients after initial surgery is only 8 months, and this patient is currently at 17 months survival post-surgery.

This increased time of patient survival over the usual time of survival post-surgery in patients such as this patient is likely due to the diagnostic testing directing administration of optimal therapeutic agents that occurred as a result of this claimed process.

The patient experience described here indicates the immense cancer therapy value of this multistep proteomic/genomic panomic approach to informing a cancer treatment decision using both the proteomic and genomic molecular status of patient tumor cells and demonstrates the extreme potential value for other cancer patients. It is critical that tissue microdissection be performed on a homogeneous population of tumor cells to achieve the most precise molecular analysis using tissue microdissection, and the presently described signal directed tissue microdissection is the cornerstone of this panomics approach to informing cancer treatment decisions.

1. A method for signal directed tissue microdissection comprising the steps of:
a) providing a DIRECTOR slide upon which is placed a section of transfer material comprising histopathologically processed or frozen tissue/cell materials characterized by cellular heterogeneity,
b) identifying particular cells, groups of cells, or sub-cellular regions to be selectively procured from the tissue by a unique inclusion signal, or signals,
c) identifying particular cells, groups of cells, or sub-cellular regions not to be selectively procured from the tissue by a unique exclusion signal, or signals, which is/are distinctly different from the inclusion signal,
d) activating a laser photon energy to strike the energy transfer coating of the DI-RECTOR slide precisely at the region, or regions, of the histopathologically processed or frozen tissue/cell materials characterized by cellular heterogeneity that display the unique inclusion signal inducing a laser induced forward transfer of those individual cells, groups of cells, or sub-cellular regions onto a receiving substrate or into a specified collection vesicle,
e) preventing activation of a laser photon energy from striking the energy transfer coating of the DIRECTOR slide at precisely the region, or regions, of the histopathologically processed or frozen tissue/cell materials characterized by cellular heterogeneity identified by the distinctly different and unique exclusion signal whereby those individual cells, groups of cells, or sub-cellular regions are specifically not transferred onto a receiving substrate or into a specified collection vesicle.

2. The method of claim 1, wherein the transfer material comprising histopathologically processed or frozen tissue/cell materials characterized by cellular heterogeneity is stained to achieve an inclusion and/or exclusion signal using non-antibody based standard histochemical stains including but not limited to hematoxylin, eosin, congo red, aldehyde fuchsin, anthraquinone derivatives, alkaline phosphatase, Bielschowsky, cajal, cresyl violet, Fontana-Masson, Giemsa, golgi stain, iron hematoxylin, luxol fast blue, luna, Mallory trichrome, Masson trichrome, Movat's pentachrome, mucicarmine, nuclear fast red, oil red O, orcien, osmium tetroxide, Papanicolaou, periodic acid-schiff, phosphotungstic acid-hematoxylin, picrosirius red, Prussian blue, reticular fiber, Romanowsky stains, safranin O, silver, sudan stains, tartrazine, toluidine blue, Van Gieson, Verhoeff, Von Kossa, and Wright's stain.

3. The method of claim 1, wherein the inclusion and/or exclusion signal results are obtained using antibody-based immunohistochemical methods.

4. The method of claim 1, wherein the inclusion and/or exclusion signal results are obtained using RNA in situ hybridization methods.

5. The method of claim 1, wherein the inclusion and/or exclusion signal is a colorimetric signal mediated by signal emission molecules.

6. The method of claim 1, wherein the inclusion and/or exclusion signal is a fluorescent signal mediated by fluorescent signal emission molecules.

7. The method of claim 1, wherein the transfer material comprising histopathologically processed or frozen tissue/cell materials characterized by cellular heterogeneity is solid tissue that is formalin fixed paraffin embedded tissue.

8. The method of claim 7, wherein the solid tissue is tumor tissue removed from a cancer patient.

9. The method of claim 7, wherein the transfer material comprising histopathologically processed or frozen tissue/cell materials characterized by cellular heterogeneity comprises a section of tissue and wherein the section is placed onto a DIRECTOR slide.

10. The method of claim 9, wherein the section is of a thickness from about 2 μM to about 50 μM.

11. The method of claim 1, wherein the particular cells, groups of cells, or sub-cellular regions are visualized using a microscope, a video camera, a video screen, a digital image, a slide scanner instrument, and/or a computer screen for the purpose of identifying and ascribing inclusion and/or exclusion signals.

12. The method of claim 11, wherein an expert in histopathology, including but not limited to a trained and licensed pathologist, identifies and ascribes through visual identification a single specific signal or multiple specific signals in particular cells, groups of cells, or sub-cellular regions as inclusion and/or exclusion signals.

13-16. (canceled)

17. The method of claim 1, wherein activation of the laser is controlled by the presence of inclusion signals and/or exclusion signals as detected and coordinated by a computer.

18-21 (canceled).

22. The method of claim 1, further comprising the step of performing a proteomic assay, or assays, on a lysate prepared from said collected cells, groups of cells, or sub-cellular regions for determining the proteomic status of said collected cells, groups of cells, or sub-cellular regions identified by an inclusion signal.

23-26. (canceled)

27. The method of claim 1, further comprising the step of performing a genomic assay, or assays, on a biochemical lysate prepared from the microdissected cells, groups of cells, or sub-cellular regions identified by an inclusion signal to be used for determining the genomic status of said collected cells, groups of cells, or sub-cellular regions identified by an inclusion signal.

28. The method of claim 27, wherein the genomic status of said collected cells, groups of cells, or sub-cellular regions identified by an inclusion signal differ from the normal DNA status as determined by differences selected from the group consisting of single nucleotide changes, multiple nucleotide changes, insertions, deletions, rearrangements, duplications, single base pair polymorphisms, transitions, transversions, inversions, copy number variations, duplications/deletions of long stretches of nucleic acids, and combinations thereof.

29. The method of claim 27, wherein said genomic assay, or assays, utilizes methodology selected from the group consisting of sequencing, Next Generation Sequencing (NGS), DNA-seq, RNA-seq, polymerase chain reaction (PCR), reverse transcription polymerase chain reaction (RT-PCR), and quantitative reverse transcription polymerase chain reaction (Q-RT-PCR).

30. The method of claim 27, wherein the genomic status of said collected cells, groups of cells, or sub-cellular regions identified by an inclusion signal differ from the normal RNA status as determined by differences that selected from the group consisting of quantitative changes in expression of single genes, quantitative changes in expression patterns of multiple genes, changes in the sequence of expressed genes, quantitative changes in RNA molecules, quantitative changes in expression patterns of RNA molecules, and changes in the sequence of expressed RNA molecules.

31. The method of claim 1, wherein the resulting proteomic and/or genomic status of said collected cells, groups of cells, or sub-cellular regions identified by an inclusion signal as determined from the assay, or assays, are used to select at least one cancer treatment strategy selected from the group consisting of standard chemotherapy agents, targeted therapeutic agents, immunomodulatory agents, and cancer vaccines.

32. The method of claim 31, wherein the proteomic and/or genomic status of said collected cells, groups of cells, or sub-cellular regions identified by an inclusion signal and preferentially microdissected is used to inform individualized cancer treatment strategy for an individual cancer patient using a multistep panomic process comprising:
a) obtaining tumor tissue from an individual cancer patient in the form of a formalin fixed paraffin embedded tissue block and placing sections from the block on DI-RECTOR slides, or receiving tissue sections previously placed on DIRECTOR slides from said block, via a physician and/or healthcare team accompanied by a test requisition form describing the requested tests,
b) isolating and collecting a highly purified population of patient tumor cells directly from said patient tumor tissue using the presently described signal directed tissue microdissection method,
c) reducing said microdissected pure population of patient tumor cells to a soluble and liquefied state,
d) developing a proteomic status of the patient's tumor cells, groups of tumor cells, or sub-cellular regions identified by an inclusion signal by detecting, quantifying, and qualifying targeted proteins and peptides in said lysate using mass spectrometry,
e) developing a genomic status of the patient's tumor cells, groups of tumor cells, or sub-cellular regions identified by an inclusion signal by analyzing nucleic acids in said lysate prepared from said microdissected tumor cells, using sequencing, next generation sequencing (NGS), PCR, RT-PCR, RNA-seq, and/or Q-RT-PCR methods to develop DNA mutation and RNA expression profiles of the patient's tumor cells,
f) informing and ascertaining an optimal cancer treatment strategy from which the patient will most likely benefit wherein said strategy is based on the panomic combination of the protein/peptide expression status, DNA mutation status, and RNA expression status obtained from said patient's signal directed microdissected tumor cells utilizing said proteomics and genomics technologies,
g) preparing a patient report containing all the protein expression, DNA mutation, and RNA expression information about the patient's tumor cells in order to convey said scientific data about said patient's tumor cells to the cancer patient's medical professional team including said patient's physician so the patient can receive an optimal treatment regimen.
